(19) United States
(12) Patent Application Publication
(10) Pub. No.: US 2020/0279621 A1
(43) Pub. Date: Sep. 3, 2020

(54) COGNITIVE ANALYSIS PROCESSING TO FACILITATE SCHEDULE PROCESSING AND SUMMARIZATION OF ELECTRONIC MEDICAL RECORDS

(71) Applicant: International Business Machines Corporation, Armonk, NY (US)

(72) Inventors: Louis Ralph Degenaro, White Plains, NY (US); Edward A. Epstein, Putnam, NY (US); Jianhe Luo, White Plains, NY (US); Jaroslaw Cwiklik, Cary, NC (US); Burn Lewis, Ossining, NY (US); Abhishek Malvankar, White Plains, NY (US)

(21) Appl. No.: 16/288,929

(22) Filed: Feb. 28, 2019

(51) Int. Cl. G16H 10/60; G06F 9/48; G06F 17/24; G06N 5/00

(52) U.S. Cl. CPC G16H 10/60; G06N 5/003; G06F 17/24; G06F 9/4887

(57) ABSTRACT

Systems, computer-implemented methods, and computer program products that can facilitate scheduling processing and summarization of an electronic medical record based on a summarization deadline are provided. According to an embodiment, a system can comprise a memory that stores computer executable components and a processor that executes the computer executable components stored in the memory. The computer executable components can comprise a task manager component that can determine a processing priority of one or more data bundles of an electronic medical record based on a summarization deadline of the electronic medical record. The computer executable components can further comprise a cognitive analysis component that can employ an artificial intelligence model to process the one or more data bundles based on the processing priority.

### BACKGROUND

The subject disclosure relates to cognitive analysis processing, and more specifically, to cognitive analysis processing to facilitate scheduling processing and summarization of electronic medical records based on a summarization deadline.

### SUMMARY

The following presents a summary to provide a basic understanding of one or more embodiments of the invention. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, systems, computer-implemented methods, or computer program products that can facilitate scheduling processing and summarization of an electronic medical record based on a summarization deadline refinement of a predicted event based on explainability data are described.

According to an embodiment, a system can comprise a memory that stores computer executable components and a processor that executes the computer executable components stored in the memory. The computer executable components can comprise a task manager component that can determine a processing priority of one or more data bundles of an electronic medical record based on a summarization deadline of the electronic medical record. The computer executable components can further comprise a cognitive analysis component that can employ an artificial intelligence model to process the one or more data bundles based on the processing priority.

According to another embodiment, a computer-implemented method can comprise determining, by a system operatively coupled to a processor, a processing priority of one or more data bundles of an electronic medical record based on a summarization deadline of the electronic medical record. The computer-implemented method can further comprise employing, by the system, an artificial intelligence model to process the one or more data bundles based on the processing priority.

According to another embodiment, a computer program product facilitating scheduling processing and summarization of an electronic medical record based on a summarization deadline is provided. The computer program product comprising a computer readable storage medium having program instructions embodied therewith, the program instructions executable by a processor to cause the processor to determine, by the processor, a processing priority of one or more data bundles of an electronic medical record based on a summarization deadline of the electronic medical record. The program instructions are further executable by the processor to cause the processor to employ, by the processor, an artificial intelligence model to process the one or more data bundles based on the processing priority.

### DESCRIPTION OF THE DRAWINGS

FIG. 1 illustrates a block diagram of an example, non-limiting system that can facilitate scheduling processing and summarization of an electronic medical record based on a summarization deadline in accordance with one or more embodiments described herein.

FIG. 2 illustrates a block diagram of an example, non-limiting system that can facilitate scheduling processing and summarization of an electronic medical record based on a summarization deadline in accordance with one or more embodiments described herein.

FIG. 3 illustrates a block diagram of an example, non-limiting system that can facilitate scheduling processing and summarization of an electronic medical record based on a summarization deadline in accordance with one or more embodiments described herein.

FIG. 4 illustrates a block diagram of an example, non-limiting system that can facilitate scheduling processing and summarization of an electronic medical record based on a summarization deadline in accordance with one or more embodiments described herein.

FIG. 5 illustrates a flow diagram of an example, non-limiting computer-implemented method that can facilitate scheduling processing and summarization of an electronic medical record based on a summarization deadline in accordance with one or more embodiments described herein.

FIGS. 6A and 6B illustrate flow diagrams of example, non-limiting computer-implemented methods that can facilitate scheduling processing and summarization of an electronic medical record based on a summarization deadline in accordance with one or more embodiments described herein.

FIG. 7 illustrates a flow diagram of an example, non-limiting computer-implemented method that can facilitate scheduling processing and summarization of an electronic medical record based on a summarization deadline in accordance with one or more embodiments described herein.

FIG. 8 illustrates a flow diagram of an example, non-limiting computer-implemented method that can facilitate scheduling processing and summarization of an electronic medical record based on a summarization deadline in accordance with one or more embodiments described herein.

FIG. 9 illustrates a block diagram of an example, non-limiting system that can facilitate scheduling processing and summarization of an electronic medical record based on a summarization deadline in accordance with one or more embodiments described herein.

FIG. 10 illustrates a flow diagram of an example, non-limiting computer-implemented method that can facilitate scheduling processing and summarization of an electronic medical record based on a summarization deadline in accordance with one or more embodiments described herein.

FIG. 11 illustrates a block diagram of an example, non-limiting operating environment in which one or more embodiments described herein can be facilitated.

FIG. 12 illustrates a block diagram of an example, non-limiting cloud computing environment in accordance with one or more embodiments of the subject disclosure.

FIG. 13 illustrates a block diagram of example, non-limiting abstraction model layers in accordance with one or more embodiments of the subject disclosure.

### DETAILED DESCRIPTION

The following detailed description is merely illustrative and is not intended to limit embodiments or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details. It is noted that the drawings of the present application are provided for illustrative purposes only and, as such, the drawings are not drawn to scale.

The field of the subject disclosure is processing of electronic medical records (EMRs) for the purpose of cognitive analysis. The problem to be solved is how to continuously ingest and reprocess EMRs for use by a cognitive analysis processing system (CAPS) in a time and processing efficient manner

As referenced herein, an electronic medical record (EMR) can represent a collection of data for one patient. An EMR can comprise one or more notes or measurements, which may be structured or unstructured. Therefore, as referenced herein, an EMR can comprise patient information in structured and unstructured form. Examples of structured EMR data can include, but are not limited to, blood pressure, pulse, temperature, respiratory rate, height, weight, red and white blood cell counts, or other structured EMR data. Examples of unstructured EMR data can include, but are not limited to, doctor's notes and observations, text from patient examinations and interviews, or other unstructured EMR data.

A cognitive analysis processing system (CAPS) can be trained with computer models (e.g., machine learning model, artificial intelligence model, etc.) that can learn from ground truth (e.g., data in EMRs) and apply what's been learned to, for example, perform question answering. A CAPS (e.g., cognitive service 404 described below) can be developed to reason about EMRs. However, the EMRs must be processed into a form that can be used by the CAPS. Patient EMRs are temporal, as medical measurements can change as time moves forward and doctor's notes are added. In addition, the CAPS may itself change, for example, new models or reasoning algorithms, which may require reprocessing of EMRs.

Inputs and requests to one or more embodiments of the subject disclosure (e.g., systems 100, 200, 300, 400, 900 described below) can comprise unprocessed patient full or partial EMRs, existing patient incremental EMRs (e.g., additional measurements, notes, etc.), or summarization inquiries (e.g., requests to summarize one or more patient EMRs). Outputs to one or more embodiments of the subject disclosure (e.g., systems 100, 200, 300, 400, 900 described in detail below) can comprise summarization results (e.g., results of a summary of an EMR).

As referenced herein, continuous ingestion can be defined as the ongoing over time receipt and processing of EMRs comprising full or partial patient records. In some embodiments, ingestion can continue until summarization is requested at which time the CAPS can reason about all the previously processed full or partial patient records previously submitted. In some embodiments, once summarization is completed, continuous ingestion can continue until the next summarization request is processed.

In some embodiments, an interface (e.g., an application programming interface (API) such as, for instance, interface component 204) and a corresponding back-end system (e.g., EMR processing and summarization system 102) are described herein to receive submissions of EMRs (e.g., full or partial EMRs) and requests for summarizations or notifications (e.g., processing status). In these embodiments, processing such EMRs and requests for summarizations of notifications (e.g., via EMR processing and summarization system 102 as described below) can be useful to, for example, question answering systems (e.g., a CAPS such as, for instance, cognitive service 404 described below) that can provide cognitive insights into patient status, possible diagnosis, or treatment.

In some embodiments, such an interface described above (e.g., an API, interface component 204) can interact with the back-end system (e.g., EMR processing and summarization system 102), which can: store EMRs; process EMRs to be in a position to respond to summarization requests; or answer requests for summarization or notifications. In some embodiments, EMRs can be stored in a database (e.g., incremental ingestion record store 412 described below) in the event that reprocessing is necessary. In some embodiments, processing of submitted EMRs and requests for summarization can involve use of cognitive analysis pipelines (e.g., cognitive analysis components 110, distributed processors, etc.).

Also, from time to time, processing errors can occur. In some embodiments, the interface and back-end system described herein can (e.g., via error handler component 202) minimize the impact of errors by isolating bad EMR data while continuing the processing of good EMR data. In some embodiments, the interface and back-end system described herein can (e.g., via error handler component 202) detect and correct bad EMR data and subsequently reprocess such corrected bad EMR data.

In some embodiments, results of processing EMRs (e.g., annotating or extracting data via cognitive analysis component 110), can also be stored in a database or object store or other persistence mechanism (e.g., in memory 104, incremental ingestion record store 412, cognitive record store 416, etc.) in preparation for summarization requests. In some embodiments, summarization processing of a patient's EMRs can be launched at various times, including on-demand by virtue of a summarization request via the interface (e.g., interface component 204), or in anticipation of a not yet received summarization request according to various heuristics including, but not limited to: receiving unprocessed or updated EMR data for a patient, knowledge of an upcoming patient visit, or the availability of back-end system resources (e.g., cognitive analysis components 110, distributed processors, etc.).

An advantage of one or more embodiments of the subject disclosure described herein (e.g., systems 100, 200, 300, 400, 900, etc.) is that ingestion processing of EMR content can be done independently of all other content. For example, deep natural language processing (NLP) of unstructured text of one note in a full patient EMR can be processed (e.g., via cognitive analysis components 110, distributed processors, etc.) independently of another note in the same EMR. Another advantage of one or more embodiments of the subject disclosure (e.g., systems 100, 200, 300, 400, 900, etc.) is that the results of such independent processing can be stored (e.g., in memory 104, incremental ingestion record store 412, cognitive record store 416, etc.) and reused (e.g., by EMR processing and summarization system 102, task manager component 108, cognitive analysis component 110, etc.) during all subsequent summarizations.

In some embodiments, explicit summarization requests are not required. For example, some or all summarization processing can occur automatically, depending on solution needs and acceptable system cost (e.g., processing cost, computation cost). In this example, scheduling of summarization processing becomes more important as it gets more expensive (e.g., computationally expensive).

In some embodiments, a representational state transfer (REST) service (e.g., REST 302 described below) can employ an interface (e.g., an API, interface component 204) of one or more embodiments of the subject disclosure to deliver requests for processing of EMRs, summarizations, or notifications. In some embodiments, a task manager (e.g., task manager component 108) can store pertinent information (e.g., metadata) pertaining to each request in a database (e.g., database (DB) 304) and can schedule one or more tasks to accomplish the desired result.

In some embodiments, the subject disclosure described herein (e.g., systems 100, 200, 300, 400, 900 described below) can segment each received EMR into independent processing bundles (e.g., data bundles 306a, 306b, 306c, 306d, 306n described below). In some embodiments, each bundle can comprise one or more pieces of structured or unstructured data. In some embodiments, such bundles can be created to optimize distributed processing performance In some embodiments, such bundles can be processed (also referred to herein as ingested) in parallel by one or more processing threads running in one or more processes deployed to one or more computation nodes (e.g., such processing can be performed via cognitive analysis component 110, distributed processors, etc.). In some embodiments, each processing thread (e.g., of cognitive analysis component 110) can comprise ingestion analytics for structured and unstructured data.

In some embodiments, task allocation (e.g., executed via task manager component 108) can comprise bundling EMRs into optimal processing collections of individual notes for processing (e.g., data bundles 306a, 306b, 306c, 306d, 306n) that can be queued and distributed (e.g., via task manager component 108) according to a scheduling algorithm (e.g., a priority scheduling algorithm, etc.).

In some embodiments, summarization of an EMR (e.g., executed via cognitive analysis component 110, distributed processors, etc.) can comprise the collection level processing of all the ingested bundles. In some embodiments, summarization of an EMR cannot begin until all the corresponding notes and measurements of the EMR (e.g., data bundles 306a, 306b, 306c, 306d, 306n) have been ingested (e.g., processed by cognitive analysis component 110, distributed processors, etc.). In some embodiments, summarizations can be queued and scheduled (e.g., via task manager component 108) based on completion of processing previously received relevant EMRs (e.g., based on processed relevant data bundles 306a, 306b, 306c, 306d, 306n).

In some embodiments, an error handler (e.g., error handler component 202) can examine the results of processing and can facilitate re-bundling or rescheduling failed processing units (e.g., data bundles 306a, 306b, 306c, 306d, 306n that could not be processed due to, for instance, a data error or a processing error). In some embodiments, the subject disclosure described herein (e.g., systems 100, 200, 300, 400, 900, etc.) can receive, store, or distribute data safely through employment of encryption (e.g., via employing a data encryption scheme such as, for instance, International Data Encryption Algorithm (IDEA), Triple Data Encryption Standard (DES), Rivest-Shamir-Adleman (RSA), Blowfish, Twofish, Advance Encryption Standard (AES), etc.).

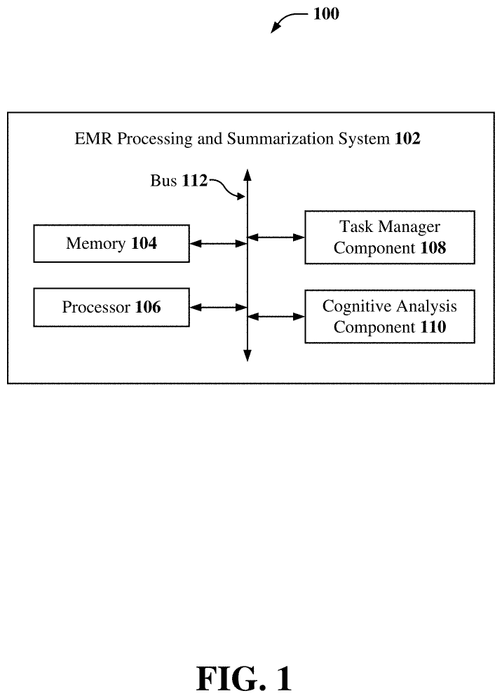

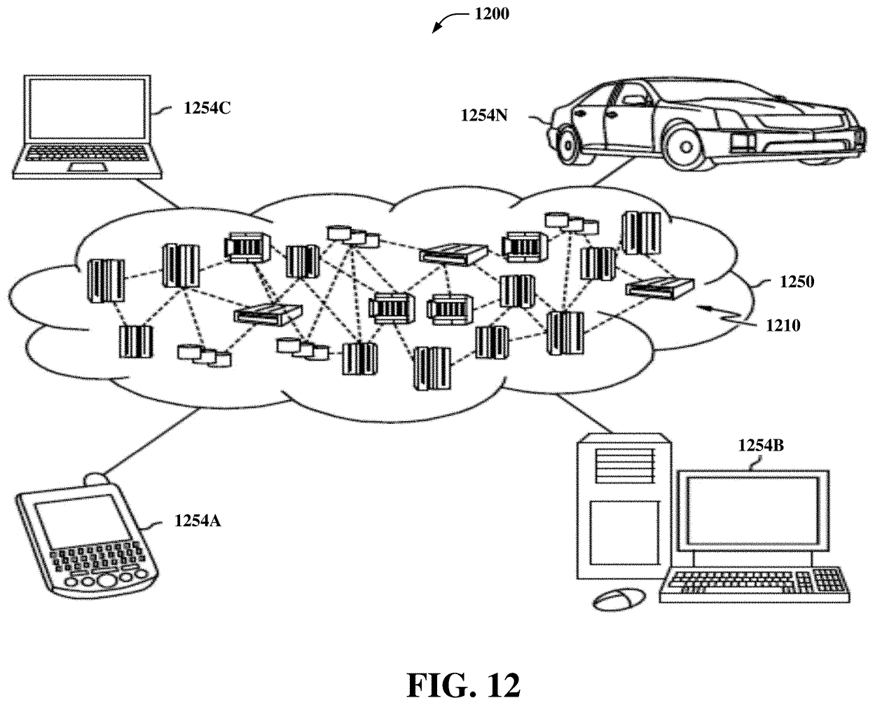

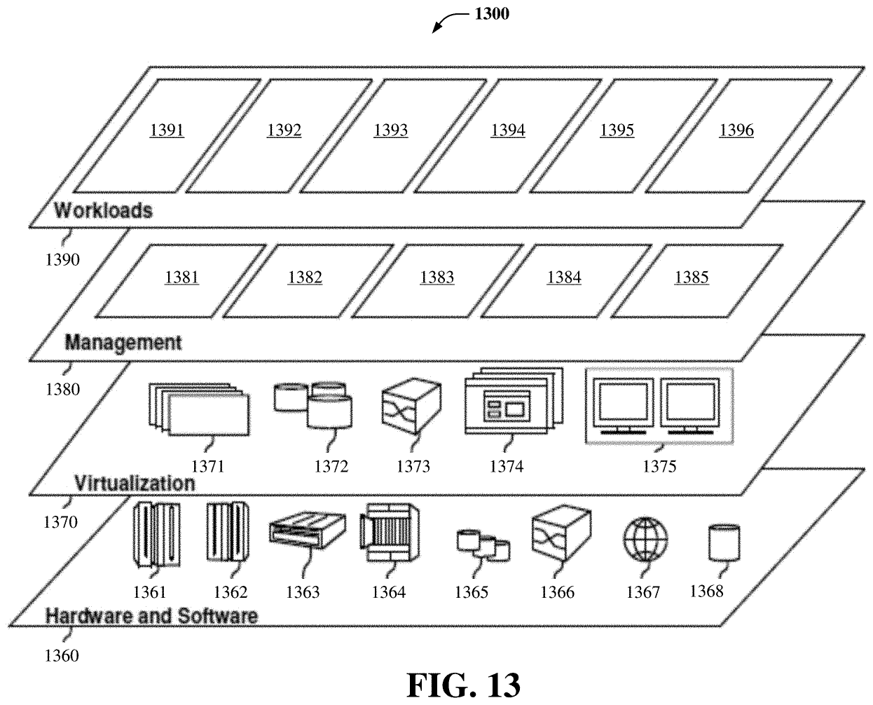

FIG. 1 illustrates a block diagram of an example, non-limiting system 100 that can facilitate scheduling processing and summarization of an electronic medical record based on a summarization deadline in accordance with one or more embodiments described herein. In some embodiments, system 100 can comprise an electronic medical record (EMR) processing and summarization system 102, which can be associated with or implemented in a cloud computing environment. For example, EMR processing and summarization system 102 can be associated with or implemented in cloud computing environment 1250 described below with reference to FIG. 12 or one or more functional abstraction layers described below with reference to FIG. 13 (e.g., hardware and software layer 1360, virtualization layer 1370, management layer 1380, or workloads layer 1390).

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Continuing now with FIG. 1. According to several embodiments, EMR processing and summarization system 102 can comprise a memory 104, a processor 106, a task manager component 108, a cognitive analysis component 110, or a bus 112.

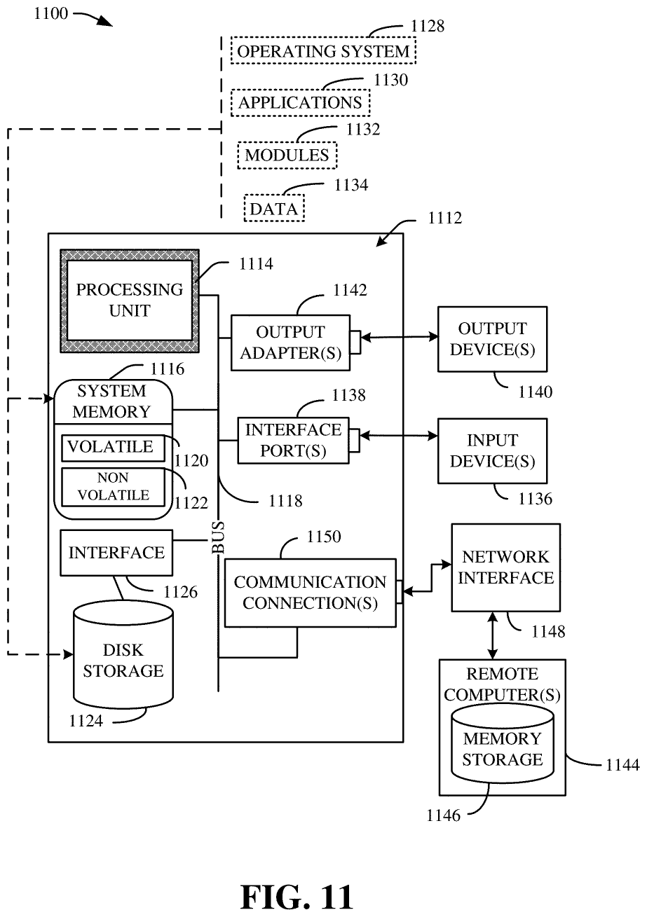

It should be appreciated that the embodiments of the subject disclosure depicted in various figures disclosed herein are for illustration only, and as such, the architecture of such embodiments are not limited to the systems, devices, or components depicted therein. For example, in some embodiments, system 100 or EMR processing and summarization system 102 can further comprise various computer or computing-based elements described herein with reference to operating environment 1100 and FIG. 11. In several embodiments, such computer or computing-based elements can be used in connection with implementing one or more of the systems, devices, components, or computer-implemented operations shown and described in connection with FIG. 1 or other figures disclosed herein.

According to multiple embodiments, memory 104 can store one or more computer or machine readable, writable, or executable components or instructions that, when executed by processor 106, can facilitate performance of operations defined by the executable component(s) or instruction(s). For example, memory 104 can store computer or machine readable, writable, or executable components or instructions that, when executed by processor 106, can facilitate execution of the various functions described herein relating to EMR processing and summarization system 102, task manager component 108, cognitive analysis component 110, or another component associated with EMR processing and summarization system 102, as described herein with or without reference to the various figures of the subject disclosure.

In some embodiments, memory 104 can comprise volatile memory (e.g., random access memory (RAM), static RAM (SRAM), dynamic RAM (DRAM), etc.) or non-volatile memory (e.g., read only memory (ROM), programmable ROM (PROM), electrically programmable ROM (EPROM), electrically erasable programmable ROM (EEPROM), etc.) that can employ one or more memory architectures. Further examples of memory 104 are described below with reference to system memory 1116 and FIG. 11. Such examples of memory 104 can be employed to implement any embodiments of the subject disclosure.

According to multiple embodiments, processor 106 can comprise one or more types of processors or electronic circuitry that can implement one or more computer or machine readable, writable, or executable components or instructions that can be stored on memory 104. For example, processor 106 can perform various operations that can be specified by such computer or machine readable, writable, or executable components or instructions including, but not limited to, logic, control, input/output (I/O), arithmetic, or the like. In some embodiments, processor 106 can comprise one or more central processing unit, multi-core processor, microprocessor, dual microprocessors, microcontroller, System on a Chip (SOC), array processor, vector processor, or another type of processor. Further examples of processor 106 are described below with reference to processing unit 1114 and FIG. 11. Such examples of processor 106 can be employed to implement any embodiments of the subject disclosure.

In some embodiments, EMR processing and summarization system 102, memory 104, processor 106, task manager component 108, cognitive analysis component 110, or another component of EMR processing and summarization system 102 as described herein can be communicatively, electrically, or operatively coupled to one another via a bus 112 to perform functions of system 100, EMR processing and summarization system 102, or any components coupled therewith. In several embodiments, bus 112 can comprise one or more memory bus, memory controller, peripheral bus, external bus, local bus, or another type of bus that can employ various bus architectures. Further examples of bus 112 are described below with reference to system bus 1118 and FIG. 11. Such examples of bus 112 can be employed to implement any embodiments of the subject disclosure.

In some embodiments, EMR processing and summarization system 102 can comprise any type of component, machine, device, facility, apparatus, or instrument that comprises a processor or can be capable of effective or operative communication with a wired or wireless network. All such embodiments are envisioned. For example, EMR processing and summarization system 102 can comprise a server device, a computing device, a general-purpose computer, a special-purpose computer, a tablet computing device, a handheld device, a server class computing machine or database, a laptop computer, a notebook computer, a desktop computer, a cell phone, a smart phone, a consumer appliance or instrumentation, an industrial or commercial device, a digital assistant, a multimedia Internet enabled phone, a multimedia players, or another type of device.

In some embodiments, EMR processing and summarization system 102 can be coupled (e.g., communicatively, electrically, operatively, etc.) to one or more external systems, sources, or devices (e.g., computing devices, communication devices, etc.) via a data cable (e.g., High-Definition Multimedia Interface (HDMI), recommended standard (RS) 232, Ethernet cable, etc.). In some embodiments, EMR processing and summarization system 102 can be coupled (e.g., communicatively, electrically, operatively, etc.) to one or more external systems, sources, or devices (e.g., computing devices, communication devices, etc.) via a network.

According to multiple embodiments, such a network can comprise wired and wireless networks, including, but not limited to, a cellular network, a wide area network (WAN) (e.g., the Internet) or a local area network (LAN). For example, EMR processing and summarization system 102 can communicate with one or more external systems, sources, or devices, for instance, computing devices (and vice versa) using virtually any desired wired or wireless technology, including but not limited to: wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Zigbee and other 802.XX wireless technologies or legacy telecommunication technologies, BLUETOOTH®, Session Initiation Protocol (SIP), ZIGBEE®, RF4CE protocol, WirelessHART protocol, 6LoWPAN (IPv6 over Low power Wireless Area Networks), Z-Wave, an ANT, an ultra-wideband (UWB) standard protocol, or other proprietary and non-proprietary communication protocols. In such an example, EMR processing and summarization system 102 can thus include hardware (e.g., a central processing unit (CPU), a transceiver, a decoder), software (e.g., a set of threads, a set of processes, software in execution) or a combination of hardware and software that facilitates communicating information between EMR processing and summarization system 102 and external systems, sources, or devices (e.g., computing devices, communication devices, etc.).

According to multiple embodiments, EMR processing and summarization system 102 can comprise one or more computer or machine readable, writable, or executable components or instructions that, when executed by processor 106, can facilitate performance of operations defined by such component(s) or instruction(s). Further, in numerous embodiments, any component associated with EMR processing and summarization system 102, as described herein with or without reference to the various figures of the subject disclosure, can comprise one or more computer or machine readable, writable, or executable components or instructions that, when executed by processor 106, can facilitate performance of operations defined by such component(s) or instruction(s). For example, task manager component 108, cognitive analysis component 110, or any other components associated with EMR processing and summarization system 102 as disclosed herein (e.g., communicatively, electronically, or operatively coupled with or employed by EMR processing and summarization system 102), can comprise such computer or machine readable, writable, or executable component(s) or instruction(s). Consequently, according to numerous embodiments, EMR processing and summarization system 102 or any components associated therewith as disclosed herein, can employ processor 106 to execute such computer or machine readable, writable, or executable component(s) or instruction(s) to facilitate performance of one or more operations described herein with reference to EMR processing and summarization system 102 or any such components associated therewith.

In some embodiments, EMR processing and summarization system 102 can facilitate performance of operations executed by or associated with task manager component 108, cognitive analysis component 110, or another component associated with EMR processing and summarization system 102 as disclosed herein. For example, as described in detail below, EMR processing and summarization system 102 can facilitate: determining a processing priority of one or more data bundles of an electronic medical record based on a summarization deadline of the electronic medical record; and/or employing an artificial intelligence model to process the one or more data bundles based on the processing priority. In other examples, as described in detail below, EMR processing and summarization system 102 can further facilitate: employing a heuristic algorithm to segment the electronic medical record into the one or more data bundles; employing a scheduling algorithm to schedule processing of the one or more data bundles based on the processing priority or summarization of the electronic medical record based on the summarization deadline, one or more processed data bundles, a processed partial electronic medical record, or a processed full electronic medical record; distributing the one or more data bundles or a summarization request to summarize the electronic medical record to one or more distributed processors; identifying at least one of a data error or a processing error; correcting the data error or the processing error; employing a scheduling algorithm to schedule processing of a corrected data bundle, the one or more data bundles, or a summarization of the electronic medical record based on a corrected data error, a corrected processing error, the processing priority, or the summarization deadline; employing a reasoning algorithm, natural language annotation, or natural language processing to perform data extraction or data annotation of data in the one or more data bundles; providing processing status of the summarization request, where the processing status comprises a completed status, a partially completed status, or a failure status; storing intermediate processing results or final processing results of the one or more data bundles or a summarization request to summarize the electronic medical record and utilizing the intermediate processing results or the final processing results to process a subsequent electronic medical record or a subsequent summarization request; or revising the processing priority of at least one data bundle in processing based on a revised summarization deadline.

According to multiple embodiments, task manager component 108 can determine a processing priority of one or more data bundles of an electronic medical record (EMR) based on a summarization deadline of the electronic medical record. For example, task manager component 108 can determine a processing priority of one or more data bundles (e.g., individual notes, bundled notes, etc.) of an EMR based on a prioritization scheme, where a summarization request with a corresponding summarization deadline can cause task manager component 108 to expedite one or more pending unprocessed data bundles corresponding to the summarization request. In this example, task manager component 108 can thereby provide at least two levels of service quality, batch and interactive, for instance.

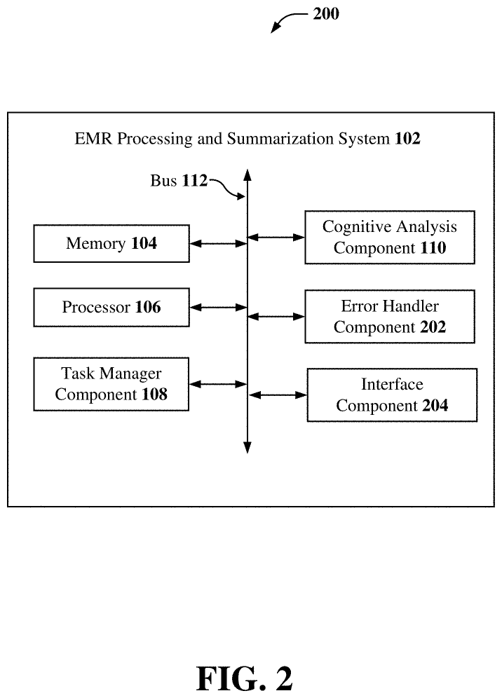

In some embodiments, task manager component 108 can determine a processing priority of one or more data bundles of an EMR based on one or more pre-scheduled summarizations, which can be pre-scheduled by task manager component 108. For example, if a patient has a visit scheduled for a specific date and time, task manager component 108 can pre-schedule a summarization for the patient that can be completed prior to the visit. In this example, such a pre-scheduled summarization of the patient's EMR can thereby dictate processing priority of one or more data bundles of the patient's EMR or one or more data bundles of another patient's EMR. In some embodiments, task manager component 108 can receive (e.g., via interface component 204 described below with reference to FIG. 2) new EMR data between the time such a pre-scheduled summarization request is scheduled by task manager component 108 and the patient visit deadline. In these embodiments, such new EMR data can be processed (e.g., via cognitive analysis component 110, distributed processors, etc.) and included in the desired summarization. In some embodiments, task manager component 108 can estimate how long a summarization will take and pre-schedule it to complete prior to the summarization deadline, thereby facilitating a summarization comprising the most up-to-date EMR data available at the time of the patient visit. In some embodiments, incomplete in-process summarizations can be interrupted or discarded by task manager component 108 if new relevant EMR data arrives.

In some embodiments, task manager component 108 can facilitate (e.g., via cognitive analysis component 110, distributed processors, etc.) summarization of an EMR in anticipation of a summarization request, even though no summarization request has been received. In some embodiments, task manager component 108 can detect that all EMR data for a patient has been processed and that there are one or more free or unused resources (e.g., cognitive analysis components 110, distributed processors, etc.) available to process summarizations. In these embodiments, task manager component 108 can pre-schedule summarization work so that a future on-demand summarization request can be immediately available.

In some embodiments, task manager component 108 can determine a processing priority of one or more data bundles of an EMR by preempting in-process work, summarization, or EMR data processing, when new high priority work arrives. For example, if a new on-demand summarization request arrives (e.g., which can be indicative of a high priority summarization request) and some or all resources (e.g., cognitive analysis component 110, distributed processors, etc.) are presently used for low priority work (e.g., work not needed to complete to meet a summarization deadline), task manager component 108 can interrupt the low priority work and re-deploy the resources to process the high priority work. In this example, by interrupting such low priority work and re-deploying the resources to process the high priority work, task manager component 108 can thereby determine processing priority of one or more data bundles of the high priority EMR or one or more data bundles of the low priority EMR.

In some embodiments, task manager component 108 can determine a processing priority of one or more data bundles based on a reprocessing scheme for the case where the ingesting pipeline analytics (e.g., cognitive analysis component 110, distributed processors, etc.) are revised or upgraded. In these embodiments, task manager component 108 (or in some embodiments, error handler component 202 as described below with reference to FIG. 2) can schedule reprocessing of the original bundles and subsequent summarizations.

In some embodiments, task manager component 108 can facilitate continuous ingestion of EMRs submitted to EMR processing and summarization system 102 via an API (e.g., interface component 204). In some embodiments, such submitted EMRs can comprise full patient records (e.g., full EMRs), for instance, when a medical enterprise (e.g., a client entity) is initializing EMR processing and summarization system 102 from its collection of patient EMRs or when a medical enterprise encounters a new patient. In some embodiments, such submitted EMRs can comprise partial patient records (e.g., partial EMRs), for instance, unprocessed notes from a recent physician visit by a patient or unprocessed test results for a patient ordered by a physician. In some embodiments, task manager component 108 can continually schedule to-be-processed new arrival of unprocessed EMRs and summarization requests. In these embodiments, task manager component 108 can further re-schedule previously scheduled but not yet started work or preempt running work accordingly to re-apportion resources (e.g., cognitive analysis component 110, distributed processors, etc.) to complete work according to summarization deadlines corresponding respectively to such work.

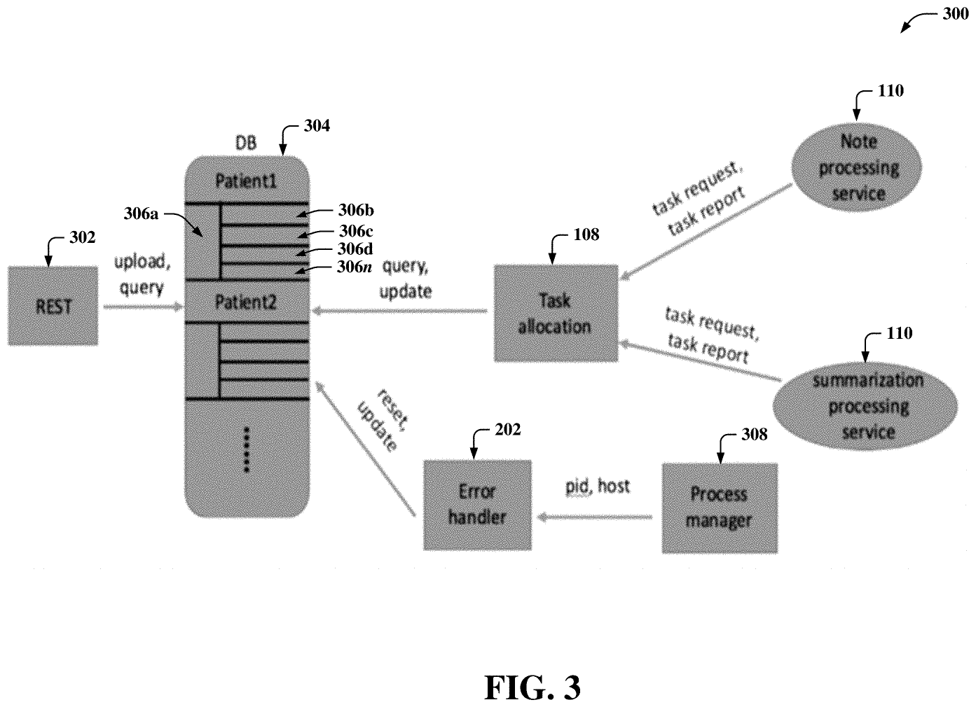

In some embodiments, task manager component 108 can employ a heuristic algorithm to segment an EMR into one or more data bundles. For example, task manager component 108 can employ a heuristic segmentation algorithm to segment an EMR into one or more data bundles comprising individual notes, bundled notes, or other EMR data that can be segmented into discrete units of data that can be independently processed (e.g., cognitive analysis component 110, distributed processors, etc.). For instance, task manager component 108 can employ a heuristic segmentation algorithm to segment an EMR into data bundles 306a, 306b, 306c, 306d, 306n illustrated in FIG. 3.

In some embodiments, interface component 204, which can comprise an API, can continually receive full and/or partial EMRs and/or summarization requests and/or prioritization requests from clients 402. In these embodiments, interface component 204 can create one or more bundles (e.g., data bundles 306a, 306b, 306c, 306d, 306n) for each EMR. In these embodiments, interface component 204 can write such bundles into a non-volatile location (e.g., in database (DB) 304) where task manager component 108 can access and schedule such bundles for processing (e.g., by cognitive analysis component 110, distributed processors, etc.). In some embodiments, bundling each EMR can be performed in such a way as to optimize one or more processing criteria including, but not limited to: least use of resources, finish processing as soon as possible, most efficient use of resources, size of individual notes, size of multi-note bundles, content of notes, content of structured data, etc. comprising heuristics. In some embodiments, EMR processing and summarization system 102 (e.g., via task manager component 108, interface component 204) can assign and/or re-assign priorities to each persisted bundle based on a summarization deadline or priority (e.g., an upcoming patient visit).

In some embodiments, task manager component 108 can employ a scheduling algorithm to schedule processing of one or more data bundles of an EMR based on a processing priority. For example, task manager component 108 can employ a scheduling algorithm including, but not limited to, a priority scheduling algorithm, where each data bundle can have a level of priority corresponding thereto (e.g., as determined by task manager component 108 as described above), a longest job first (LJF) scheduling algorithm, a modified version of a shortest job first (SJF) scheduling algorithm (e.g., also known as shortest job next (SJN) or shortest process next (SPN)), or another scheduling algorithm. For instance, task manager component 108 can employ a priority scheduling algorithm to select from a run queue (e.g., in memory 104, database (DB) 304, incremental ingestion record store 412, etc.) a data bundle having the highest level of priority corresponding thereto (e.g., as determined by task manager component 108 as described above).

In some embodiments, task manager component 108 can employ a scheduling algorithm to schedule summarization of an EMR based on: a summarization deadline; one or more processed data bundles; a processed partial EMR; or a processed full EMR. For instance, task manager component 108 can employ a priority scheduling algorithm to select from a run queue (e.g., in memory 104, database (DB) 304, incremental ingestion record store 412, etc.) a summarization request (e.g., a summarization task) having the highest level of priority corresponding thereto (e.g., as determined by task manager component 108 based on a corresponding summarization deadline as described above).

In some embodiments, task manager component 108 can distribute one or more data bundles or a summarization request to summarize an EMR to one or more distributed processors. For example, task manager component 108 can distribute (e.g., via bus 112 or a network such as, for instance, the Internet) one or more data bundles or a summarization request to summarize an EMR to one or more cognitive analysis components 110, which can comprise cognitive analytics pipelines, distributed processors (e.g., virtual processors), independent central processing units (CPUs), or other distributed processors that can process such one or more data bundles or summarization requests.

In some embodiments, task manager component 108 can track processing status of one or more data bundles of an EMR or summarization status of the EMR. For example, task manager component 108 can record information (e.g., metadata) related to each data bundle processing task or summarization task in a database (e.g., memory 104, database (DB) 304, incremental ingestion record store 412, etc.), where such information can comprise data related to processing status of each task (e.g., pending, in processing, completed, failed, etc.). For instance, upon distribution of one or more data bundle processing tasks or summarization tasks, task manager component 108 can record identification information of the distributed processors executing such tasks. In this example, upon completion (or failure) of such tasks, the distributed processor can report to task manager component 108 the status of such task (e.g., completed, failed, etc.).

In some embodiments, task manager component 108 can revise the processing priority of at least one data bundle in processing based on a revised summarization deadline. For example, task manager component 108 can revise a processing priority of at least one data bundle of an EMR and preempt in-process work, summarization, or EMR data processing based on a revised summarization deadline. For instance, if one or more data bundles are in processing and a summarization deadline corresponding to one or more other data bundles is revised (e.g., revised to a shorter deadline, closer in time), task manager component 108 can revise the processing priority of the one or more data bundles currently in processing (e.g., to a lower level of priority) and preempt processing of such data bundles in favor of processing the other data bundles corresponding to the revised summarization deadline. Similarly, in some embodiments, if a patient visit has been canceled or re-scheduled for a later time, task manager component 108 can revise the processing priority of the previously scheduled one or more associated data bundles and summary request (e.g., to a higher level of priority) so as to utilize resources for higher priority work.

According to multiple embodiments, cognitive analysis component 110 can employ an artificial intelligence model to process the one or more data bundles based on the processing priority. In some embodiments, cognitive analysis component 110 can comprise a computing device (e.g., CPU, distributed processor, virtual processor, etc.). In some embodiments, cognitive analysis component 110 can comprise or employ an artificial intelligence model including, but not limited to, a classification model, a probabilistic model, statistical-based model, an inference-based model, a deep learning model, a neural network, long short-term memory (LSTM), fuzzy logic, expert system, Bayesian model, or another model that can process one or more data bundles of an EMR based on a processing priority (e.g., as determined by task manager component 108 as described above). For example, cognitive analysis component 110 can comprise an artificial intelligence model that can employ a reasoning algorithm, natural language annotation, or natural language processing to perform data extraction or data annotation of data in one or more data bundles of an EMR.

In some embodiments, cognitive analysis component 110 can be trained (e.g., explicitly or implicitly based on a plurality of EMRs or data bundles thereof) at different levels and operate at different levels. For example, a level 1 version of the pipeline analytics (e.g., cognitive analysis component 110, distributed processors, etc.) can be deployed at one point in time yielding a summarization for a patient that is repeatable. In this example, at a different point in time, a level 2 version of the pipeline analytics (e.g., cognitive analysis component 110, distributed processors, etc.) can be deployed also yielding a summarization for a patient that is repeatable. In these examples, the level 1 and level 2 summarizations, however, can differ.

In some embodiments, EMR processing and summarization system 102 can connect (e.g., via a network such as, for instance, the Internet) to one or more peer systems to share the ingested raw data (e.g., a full EMR, partial EMR, data bundles, etc.) or annotated data (e.g., processed EMRs or data bundles). In one embodiment, peer systems can employ such data for training of future levels of EMR processing and summarization system 102 to effect improvements in, for example, the accuracy of and speed to obtain summarizations.

In some embodiments, EMR processing and summarization system 102 (e.g., via cognitive analysis component 110) can be scalable or adaptable. For example, task manager component 108 can provide prioritized work as individual notes, bundled notes, or summarizations that need to be performed to one or more distributed, independent processors (e.g., cognitive analysis component 110, distributed processors, etc.) located on one or more distributed central processing units (CPUs). In this example, each distributed processor can process notes only, summarizations only, or either. In some embodiments, the total number of such distributed processors can be increased or decreased dynamically, based on demand and availability of resources. In some embodiments, the balance between notes processing and summarization processing can also be dynamically changed (e.g., via EMR processing and summarization system 102, interface component 204, etc.).

In some embodiments, EMR processing and summarization system 102 (e.g., via cognitive analysis component 110) can work in a reliable pull-based mode which helps to increase the throughput by delivering work as soon as it is completed by a worker (e.g., a distributed processor) which can be multi-threaded (e.g., multiple processing threads). In some embodiments, such a pull-based mode can also decrease the overall latency of EMR processing and summarization system 102.

FIG. 2 illustrates a block diagram of an example, non-limiting system 200 that can facilitate scheduling processing and summarization of an electronic medical record based on a summarization deadline in accordance with one or more embodiments described herein. In some embodiments, system 200 can comprise EMR processing and summarization system 102. In some embodiments, EMR processing and summarization system 102 can comprise an error handler component 202 or an interface component 204. Repetitive description of like elements or processes employed in respective embodiments is omitted for sake of brevity.

According to multiple embodiments, error handler component 202 can: detect a data error or a processing error; correct the data error or the processing error; or employ a scheduling algorithm (e.g., a priority scheduling algorithm) to schedule processing of a corrected data bundle (e.g., a re-bundled data bundle), one or more original data bundles, or a summarization of an EMR based on a corrected data error (e.g., a re-bundled data bundle), a corrected processing error (e.g., an updated cognitive analysis model of a distributed processor), processing priority, or a summarization deadline. In some embodiments, error handler component 202 can minimize the impact of data or processing errors by isolating bad EMR data while continuing the processing of good EMR data. In some embodiments, error handler component 202 detect and correct bad EMR data and subsequently reprocess such corrected bad EMR data. In some embodiments, error handler component 202 can examine the results of processing and can facilitate re-bundling or rescheduling failed processing units (e.g., data bundles that could not be processed due to, for instance, a data error or a processing error).

According to multiple embodiments, interface component 204 can: receive an EMR or a summarization request to summarize the electronic medical record; and provide processing status of the summarization request, where the processing status comprises a completed status, a partially completed status, or a failure status. In some embodiments, interface component 204 can comprise an application programming interface (API) that can serve as an interface between a CAPS (e.g., REST 302 or cognitive service 404 as described below) and EMR processing and summarization system 102

In some embodiments, interface component 204 can answer queries (e.g., from a client entity) for completed and pending work. For example, interface component 204 can answer queries including, but not limited to: get list of “recently” completed EMRs; get list of “recently” completed summarizations; get list of active EMRs; get list of active summarizations; get list of pending EMRs; get list of pending summarizations; delete active or pending EMR; delete active or pending summarization; re-prioritize summarization (e.g. for sooner or later); add EMR for processing; add summarization of processing; or another query.

In some embodiments, EMR processing and summarization system 102 (e.g., via interface component 204) can take full or partial EMRs and make bundles (e.g., data bundles scheduled for processing by task manager component 108), which can comprise non-overlapping subsets of notes that can be deposited (e.g., via interface component 204 or task manager component 108) directly into the persistent store (e.g., memory 104, database (DB) 304, incremental ingestion record store 412, cognitive record store 416, etc.) with a priority assigned thereto, which task manager component 108 can use to distribute for processing. In some embodiments, each bundle can be independently processed (e.g., cognitive analysis component 110, distributed processors, etc.). In these embodiments, a corresponding summarization can be submitted at the same time by interface component 204 or subsequently. In some embodiments, task manager component 108 does not schedule summarization processing until all the notes (e.g., data bundles) upon which the summarization depends have been processed.

In some embodiments, interface component 204 can provide a callback facility where, upon completion of processing a submitted EMR summarization request, interface component 204 can notify the submitter (e.g., a client entity) indicating that the work has been completed. In some embodiments, such notification can include the results or errors that occurred during processing of notes (e.g., data bundles) or summarization.

In some embodiments, EMR processing and summarization system 102 (e.g., via error handler component 202, interface component 204, etc.) can facilitate summarizations that indicate specific EMR data that has not been considered in the summarization due to, for example, errors during processing or late arrival. In these embodiments, EMR processing and summarization system 102 (e.g., via error handler component 202, interface component 204, etc.) can employ an interface (e.g., an API) to clearly present to a client entity (e.g., a computing device operated by a human end user) which EMR data (e.g., data bundles) have been considered in the summarization and which EMR data have not been considered in the summarization.

FIG. 3 illustrates a block diagram of an example, non-limiting system 300 that can facilitate scheduling processing and summarization of an electronic medical record based on a summarization deadline in accordance with one or more embodiments described herein. In some embodiments, system 300 can comprise system 100, system 200, or EMR processing and summarization system 102. Repetitive description of like elements or processes employed in respective embodiments is omitted for sake of brevity.

In some embodiments, system 300 can comprise a representational state transfer (REST) 302, a database (DB) 304, a task allocation (e.g., task manager component 108), a note processing service (e.g., cognitive analysis component 110, distributed processors, etc.), a summarization processing service (e.g., cognitive analysis component 110, distributed processors, etc.), an error handler (e.g., error handler component 202), or a process manager 308. In some embodiments, database (DB) 304 can comprise one or more data bundles 306a, 306b, 306c, 306d, 306n (where n represents a total quantity of data bundles).

In some embodiments, REST 302 can employ an interface (e.g., an API, interface component 204) to deliver requests for processing of EMRs, summarizations, or notifications. In some embodiments, a task allocation (e.g., task manager component 108) can store pertinent information (e.g., metadata) pertaining to each request in database (DB) 304 and can schedule one or more tasks to accomplish the desired result.

In some embodiments, interface component 204 or task allocation (e.g., task manager component 108) can segment each received EMR into independent processing data bundles 306a, 306b, 306c, 306d, 306n. In some embodiments, each data bundle 306a, 306b, 306c, 306d, 306n can comprise one or more pieces of structured or unstructured data. In some embodiments, such bundles can be created to optimize distributed processing performance and stored in database (DB) 304. In some embodiments, such data bundles can be processed (also referred to herein as ingested) in parallel by one or more processing threads running in one or more processes deployed to one or more computation nodes of note processing service or summarization processing service (e.g., cognitive analysis component 110, distributed processors, etc.). In some embodiments, each processing thread (e.g., of cognitive analysis component 110) can comprise ingestion analytics for structured and unstructured data.

In some embodiments, data bundles 306a, 306b, 306c, 306d, 306n can be queued and distributed by task allocation (e.g., task manager component 108) to note processing service or summarization processing service (e.g., cognitive analysis component 110, distributed processors, etc.) according to a scheduling algorithm (e.g., a priority scheduling algorithm, etc.).

In some embodiments, summarization of an EMR (e.g., executed via cognitive analysis component 110, distributed processors, etc.) can comprise the collection level processing of all the ingested data bundles 306a, 306b, 306c, 306d, 306n). In some embodiments, summarization of an EMR cannot begin until all the corresponding notes and measurements of the EMR (e.g., data bundles 306a, 306b, 306c, 306d, 306n) have been ingested (e.g., processed) by note processing service or summarization processing service (e.g., cognitive analysis component 110, distributed processors, etc.). In some embodiments, summarizations can be queued and scheduled (e.g., via task manager component 108) based on completion of processing previously received relevant EMRs (e.g., based on processed relevant data bundles 306a, 306b, 306c, 306d, 306n). In some embodiments, processing of an EMR can be expedited by re-using one or more previous ingestions together with scheduling of newly arrived not-yet-ingested portions of the EMR.

In some embodiments, an error handler (e.g., error handler component 202) can examine the results of processing and can facilitate re-bundling or rescheduling failed processing units (e.g., data bundles 306a, 306b, 306c, 306d, 306n that could not be processed due to, for instance, a data error or a processing error).

In some embodiments, process manager 308 can instantiate one or more EMR processing or summarization tasks. In some embodiments, process manager 308 can comprise an entity (e.g., a computing device, a controller, a remote processing unit, etc.) that can be queried by task allocation (e.g., task manager component) 108 to identify one or more distributed processors (e.g., one or more cognitive analysis components 110) executing an EMR processing or summarization task.

In some embodiments (e.g., in a full or incremental patient records submitted scenario), REST 302 can call (e.g., via interface component 204) EMR processing and summarization system 102 to chunk (e.g., segment via task manager component 108) a patient's EMR data into multiple tasks (e.g., to aggregate several notes together as one single task). In these embodiments, REST 302 can call (e.g., via interface component 204) EMR processing and summarization system 102 to submit such tasks to database (DB) 304 and set their status to “READY”. In these embodiments, note processing service and summarization processing service (e.g., cognitive analysis component 110, distributed processors, etc.) can continuously send requests to task allocation (e.g., task manager component 108). In these embodiments, task allocation (e.g., task manager component 108) can query for available tasks from database (DB) 304 according to the types (e.g., note task, summarization task, etc.) and return tasks back to relative services (e.g., note processing service or summarization processing service). In these embodiments, once the task is assigned to a specific service (e.g., note processing service or summarization processing service), task allocation (e.g., task manager component 108) can update its status to “PROCESSING” as well as update “service_id” column to reflect the identification of the specific service executing the task (e.g., the specific distributed processor). In these embodiments, when the service (e.g., note processing service or summarization processing service) finishes the task, it can send a task report back to task allocation (e.g., task manager component 108), which can update the task status from “PROCESSING” to “FINISHED”.

In some embodiments (e.g., in a submit summarization request scenario), REST 302 can call interface component 204 to submit a summarization request. In these embodiments, REST 302 can call interface component 204 to generate a summarization task in database (DB) 304 and set the summarization task status to “SCHEDULE”. In these embodiments, when all tasks submitted previously that correspond to a certain patient are finished (e.g., processing of all related data bundles), interface component 204 or task manager component 108 can set the summarization task to “READY”, allowing task allocation (e.g., task manager component 108) to assign this available summarization task to next summarization process request for work (e.g., tasks submitted for this patient after the summarization request will be ignored for the summarization at hand).

In some embodiments (e.g., in a time out exception scenario), error handler component 202 can query tasks in database (DB) 304 at short intervals to check if current time minus process time is larger than allow-maximum-process-time. In these embodiments, REST 302, interface component 204, or task allocation (e.g., task manager component 108) can set the status of the task to “TIMEOUT”.

In some embodiments (e.g., in a service down scenario), process manager 308 can detect when a service is down (e.g., note processing service or summarization processing service) and can restart the service again. In these embodiments, process manager 308 can send (e.g., at the time process manager 308 restarts the service) pid and host of the service to error handler component 202. In these embodiments, error handler component 202 can reset the task assigned to that service to be “READY” again.

In some embodiments (e.g., in an aggregation task process error scenario), when a service (e.g., note processing service or summarization processing service) has errors in processing aggregation task, such service can report to task allocation (e.g., task manager component 108) and task allocation can separate aggregation task into several individual tasks based on notes. In these embodiments, task allocation (e.g., task manager component 108) can get “submit-time”, “max-duration”, and “max-time” for aggregation task and delete aggregation task entry in database (DB) 304. In these embodiments, task allocation (e.g., task manager component 108) can respectively submit several individual tasks to database (DB) 304 with original “submit-time”, “max-duration”, “max-time”.

In some embodiments (e.g., in a single note task process error scenario), when a service (e.g., note processing service or summarization processing service) has an error in processing a single note task, such service can report to task allocation (e.g., task manager component 108) and task allocation can set the status of the single note task to “ERROR”. In these embodiments, REST 302 can call interface component 204 to update content of task and reset status to “READY” again.

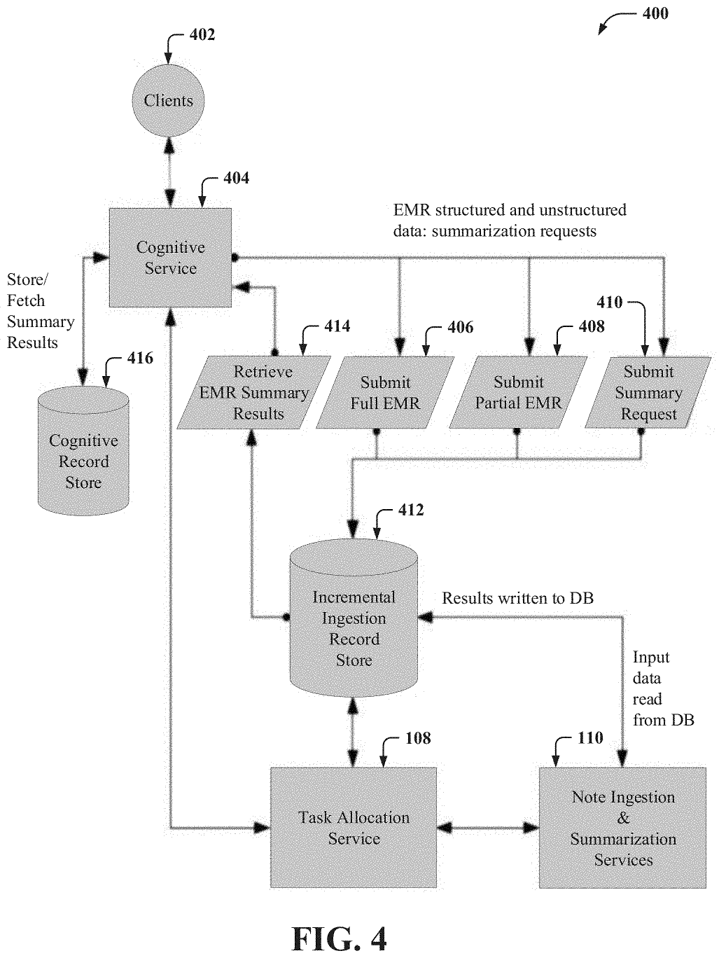

FIG. 4 illustrates a block diagram of an example, non-limiting system 400 that can facilitate scheduling processing and summarization of an electronic medical record based on a summarization deadline in accordance with one or more embodiments described herein. In some embodiments, system 400 can comprise EMR processing and summarization system 102. In some embodiments, FIG. 4 can illustrate data access and processing paths employing the ingestion interface to the back-end system of the subject disclosure. Repetitive description of like elements or processes employed in respective embodiments is omitted for sake of brevity.

In some embodiments, clients 402 can comprise one or more remote computing devices, which can be operated by a human end user. In some embodiments, cognitive service 404 can comprise REST 302 described above with reference to FIG. 3 and an application programming interface (API), neither of which are illustrated in FIG. 4. In some embodiments, such an API can comprise interface component 204 described above with reference to FIG. 2, which can facilitate communicating one or more full EMR 406, partial EMR 408, summary request 410, or EMR summary results 414 between cognitive service 404 (e.g., REST 302) and incremental ingestion record store 412. In some embodiments, incremental ingestion record store 412 can comprise memory 104 described above with reference to FIG. 1. In some embodiments, cognitive record store 416 can comprise a memory component that can be the same or similar type of memory as described herein for memory 104. In some embodiments, the task allocation service denoted in FIG. 4 can comprise task manager component 108 described above with reference to FIG. 1. In some embodiments, the note ingestion and summarization services denoted in FIG. 4 can comprise cognitive analysis component 110 described above with reference to FIG. 1.

In some embodiments, with respect to processing EMRs, one or more patient records (e.g., full EMR 406, partial EMR 408) or summary requests (e g , summary request 410) can be received (e.g., from clients 402) by system 400 (e.g., via cognitive service 404) and can be stored in an ingestion record base (e.g., incremental ingestion record store 412). In some embodiments, newly arrived unprocessed records can be marked (e.g., via task manager component 108) as “pending”. In some embodiments, task manager component 108 can bundle EMRs (e.g., full EMR 406, partial EMR 408) and can schedule these pending records for processing (e.g., by cognitive analysis component 110). In some embodiments, task manager component 108 can distribute or cognitive analysis component 110 can select records (e.g., full EMR 406, partial EMR 408, or data bundles thereof) for analytics pipeline annotating (e.g., processing) and mark same as “working”. In some embodiments, if processing completes successfully, such records results can be stored (e.g., via cognitive service 404) in a cognitive record base (e.g., cognitive record store 416) and can be marked as ‘completed’, otherwise records can be marked as ‘error’. In some embodiments, such error records can be re-bundled or rescheduled (e.g., via task manager component 108).

In some embodiments, with respect to processing a summarization (e.g., summarization request to summarize a patent's EMR), a patient summarization request (e.g., summary request 410) can be received (e.g., from clients 402) by system 400 (e.g., via cognitive service 404) and can be stored in incremental ingestion record store 412. In some embodiments, task manager component 108 (e.g., denoted as task allocation service in FIG. 4) can schedule the summarization pending the completion of an already received patient EMR (e.g., pending complete processing of one or more data bundles of the patient EMR). In some embodiments, the summarization can be processed by a corresponding analytics pipeline (e.g., cognitive analysis component 110, denoted as note ingestion and summarization services in FIG. 3) and the results can be stored in a cognitive record base (e.g., cognitive record store 416).

In some embodiments, with respect to queries (e.g., processing status queries), system 400 (e.g., via task manager component 108) can report (e.g., to cognitive service 404) the processing status of submitted EMRs and submitted request for summarization, which can include detailed error stack tracing (e.g., provided by error handler component 202). In some embodiments, such queries can be for a single EMR status or summarization or a list thereof with corresponding results returned (e.g., via task manager component 108).

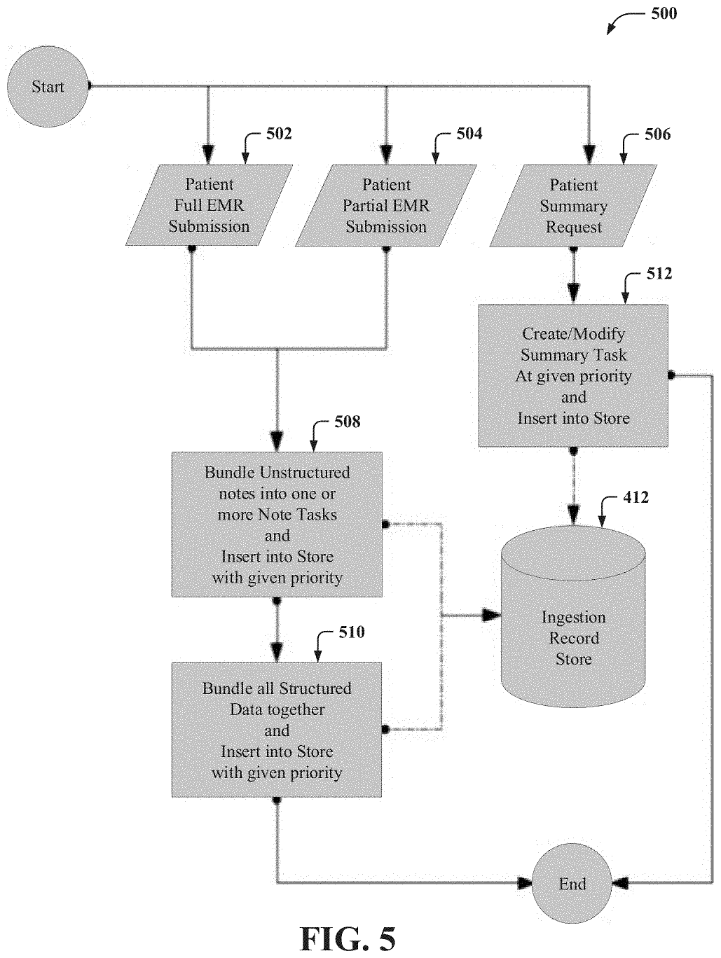

FIG. 5 illustrates a flow diagram of an example, non-limiting computer-implemented method 500 that can facilitate scheduling processing and summarization of an electronic medical record based on a summarization deadline in accordance with one or more embodiments described herein. In some embodiments, FIG. 5 can illustrate steps to receive and schedule a full or partial EMR or a summary request by task manager component 108. Repetitive description of like elements or processes employed in respective embodiments is omitted for sake of brevity.

In some embodiments, at 502, task manager component 108 can receive (e.g., via cognitive service 404) a request to process a full EMR for a patient (e.g., full EMR 406). At 504, task manager component 108 can receive (e.g., via cognitive service 404) a request to process a partial EMR for a patient (e.g., partial EMR 408). At 506, task manager component 108 can receive (e.g., via cognitive service 404) a summary request for a patient (e.g., summary request 410).

In some embodiments, if task manager component 108 receives an EMR, at 508 and 510, task manager component 108 can apply a pluggable heuristics algorithm to break up the processing request into pieces (e.g., data bundles 306a, 306b, 306c, 306d, 306n) such that multiple pieces can be processed in parallel (e.g., simultaneously). For example, at 508, task manager component 108 can apply a pluggable heuristics algorithm to bundle unstructured notes (e.g., data in an EMR) into one or more note tasks and at 510, task manager component 108 can apply a pluggable heuristics algorithm to bundle structured data together. In some embodiments, at 508 and 510, task manager component 108 can further add the bundles to the end of a queue comprising queued work for processing. For instance, at 508 and 510, task manager component 108 can insert the bundles into incremental ingestion record store 412 with a processing priority assigned to each bundle.

In some embodiments, if task manager component 108 receives a summary request, at 512, task manager component 108 can determine the summarization deadline corresponding to the summary request, where such summarization deadline can comprise a deadline for processing. In some embodiments, such summarization deadline can be assigned by, for example, clients 402, based on a patient scheduled appointment date and time. In some embodiments, at 512, task manager component 108 can insert the summary request into the queue for processing according to a processing priority assigned to the summary request that can be based on the summarization deadline. Additionally or alternatively, in some embodiments, at 512, any EMRs (e.g., full EMR 406, partial EMR 408, data bundles 306a, 306b, 306c, 306d, 306n, etc.) that are prerequisite to the summary request (e.g., must be processed before the summary request) can be moved up in the queue (e.g., via task manager component 108) such that processing of the EMRs can be completed before the summary request is processed.

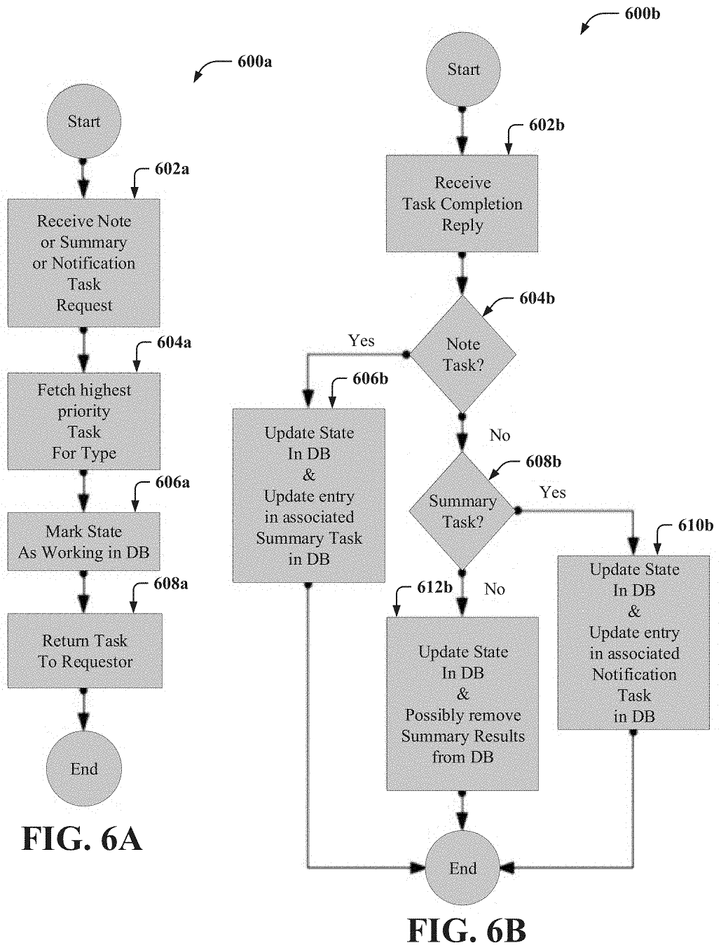

FIGS. 6A and 6B illustrate flow diagrams of example, non-limiting computer-implemented methods 600a, 600b that can facilitate scheduling processing and summarization of an electronic medical record based on a summarization deadline in accordance with one or more embodiments described herein. In some embodiments, FIGS. 6A and 6B can illustrate steps to process a full or partial EMR or a summary request that can be received and scheduled by task manager component 108. Repetitive description of like elements or processes employed in respective embodiments is omitted for sake of brevity.

In some embodiments, at 602a (FIG. 6A), task manager component 108 can receive a request for processing from an available cognitive analytics pipeline (e.g., cognitive analysis component 110, one or more distributed processors, etc.). For example, at 602a, task manager component 108 can receive a note task request (e.g., a request to process an EMR or data bundle thereof), a summary task request (e.g., a request to summarize an EMR), or a notification task request (e.g., a processing status query) from cognitive analysis component 110 or one or more distributed processors available to perform such tasks. In some embodiments, at 604a, task manager component 108 can retrieve the highest priority work item (e.g., highest priority note task or highest priority summary task), if any. In some embodiments, at 606a, task manager component 108 can mark the state of such highest priority work item as ‘working’ in database (DB) 304. In some embodiments, at 608a, task manager component 108 can deliver to such cognitive analytics pipeline the highest priority work item (e.g., highest priority note task or highest priority summary task), if any.

In some embodiments, at 602b (FIG. 6B), task manager component 108 can receive a task completion reply. For example, task manager component 108 can receive a note task completion reply, a summary task completion reply, or a notification task completion reply from cognitive analysis component 110 or one or more distributed processors. In some embodiments, at 604b, task manager component 108 can determine if the task completion reply comprises a note task completion reply. In some embodiments, if yes, at 606b, task manager component 108 can update the state of such note task in database (DB) 304 and update entry in associated summary task in database (DB) 304. In some embodiments, if no, at 608b, task manager component 108 can determine if the task completion reply comprises a summary task completion reply. In some embodiments, if yes, at 610b, task manager component 108 can update the state of such summary task in database (DB) 304 and update entry in associated notification task in database (DB) 304. In some embodiments, if no, at 612b, task manager component 108 can update state of such task in database (DB) 304 and remove summary results from database (DB) 304.

In some embodiments, task manager component 108 can assess the state of delivered work. For example, task manager component 108 can mark a timed-out work item as ‘pending’ and can later deliver such time-out work item to an alternative pipeline. In another example, task manager component 108 can retire a failed work item by de-bundling and scheduling the individual notes for processing. In some embodiments, task manager component 108 can mark atomic notes as ‘error’.

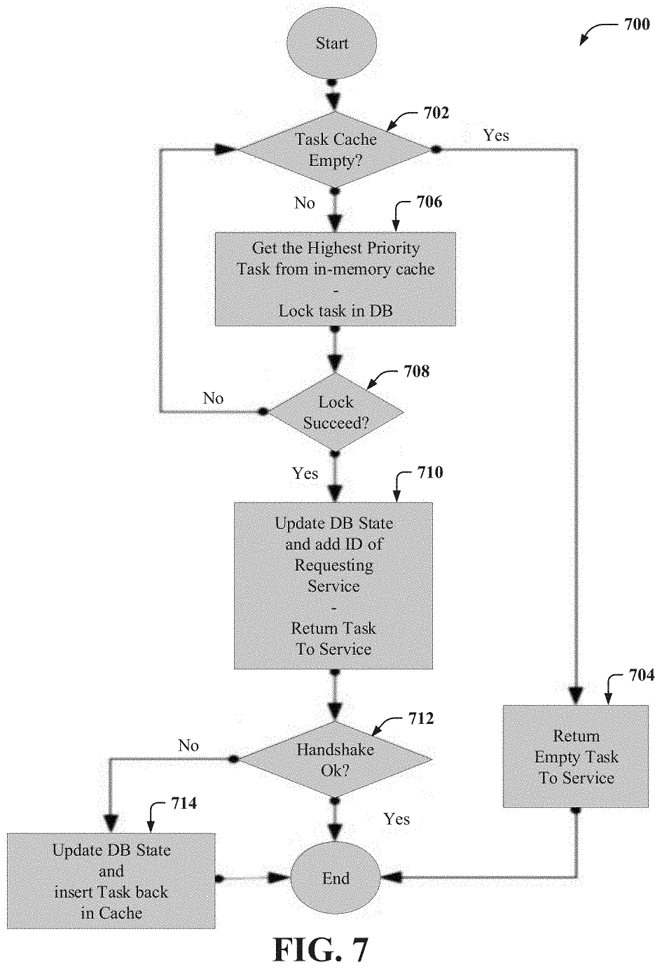

FIG. 7 illustrates a flow diagram of an example, non-limiting computer-implemented method 700 that can facilitate scheduling processing and summarization of an electronic medical record based on a summarization deadline in accordance with one or more embodiments described herein. In some embodiments, FIG. 7 can illustrate steps to process a full or partial EMR or a summary request by an analytics pipeline worker (e.g., cognitive analysis component 110, one or more distributed processors, etc.). Repetitive description of like elements or processes employed in respective embodiments is omitted for sake of brevity.

In some embodiments, a cognitive analytics pipeline worker (e.g., cognitive analysis component 110, one or more distributed processors, etc.) can request a work item from task manager component 108. In some embodiments, if a work item (e.g., a note task, summary task, etc.) is provided by task manager component 108, such a cognitive analytics pipeline worker (also referred to herein as worker) can be employed to process the work item and if processing completes successfully, the results can be stored in a memory component (e.g., memory 104, database (DB) 304, incremental ingestion record store 412, cognitive record store 416, etc.). In some embodiments, the worker can report success or failure (e.g., handshake success or failure, processing success or failure, etc.) to task manager component 108.

In some embodiments, at 702, task manager component 108 can determine if a task cache of task manager component 108 is empty. In some embodiments, if yes, at 704, task manager component 108 can return empty task to service (e.g., to such cognitive analytics pipeline worker). In some embodiments, if no, at 706, task manager component 108 can retrieve the highest priority task from in-memory cache and lock the task in database (DB) 304. In some embodiments, at 708, task manager component 108 can determine if the lock succeeded. In some embodiments, if no, method 700 can return to step 702. In some embodiments, if yes, at 710, task manager component 108 can update database (DB) 304 state, add identification (ID) of requesting service (e.g., ID of a distributed processor), and return the task to the service (e.g., to the cognitive analytics pipeline worker). In some embodiments, at 712, task manager component 108 can determine if the handshake (e.g., handoff of task to a distributed processor) is successful. In some embodiments, if yes, method 700 ends. In some embodiments, if no, at 714, task manager component 108 can update database (DB) 304 and insert task back into cache.

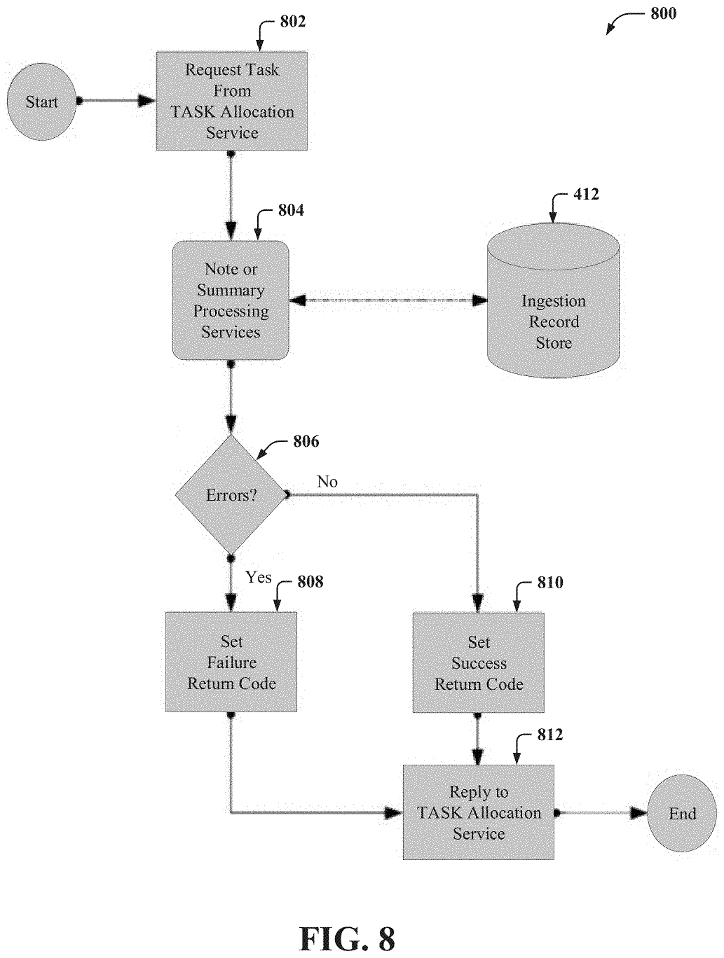

FIG. 8 illustrates a flow diagram of an example, non-limiting computer-implemented method 800 that can facilitate scheduling processing and summarization of an electronic medical record based on a summarization deadline in accordance with one or more embodiments described herein. In some embodiments, FIG. 8 can illustrate steps to process a full or partial EMR or a summary request by an analytics pipeline worker (e.g., cognitive analysis component 110, one or more distributed processors, etc.). Repetitive description of like elements or processes employed in respective embodiments is omitted for sake of brevity.

In some embodiments, at 802, one or more cognitive analytics pipeline workers (e.g., cognitive analysis component 110, one or more distributed processors, etc.) can request a task (e.g., a work item such as, for example, a note task, a summary task, etc.) from task allocation service (e.g., task manager component 108). In some embodiments, at 804, note or summary processing services (e.g., execution of a task, for example, a note task, a summary task, etc.) can be performed by the cognitive analytics pipeline worker. In some embodiments, at 804, the cognitive analytics pipeline worker can receive (e.g., from task manager component 108) or pull the task having the highest processing priority from incremental ingestion record store 412 (e.g., denoted ingestion record store in FIG. 8) and execute (or attempt to execute) such task.

In these embodiments, at 806, the cognitive analytics pipeline worker or error handler component 202 can determine whether an error has occurred (e.g., a data error identified in the EMR, an error that occurred during bundling of the EMR data, an error that occurred during processing of a data bundle or a summary request, etc.). In some embodiments, if an error has occurred, at 808, the cognitive analytics pipeline worker or error handler component 202 can set a failure return code and at 812, the cognitive analytics pipeline worker or error handler component 202 can reply to task allocation service (e.g., task manager component 108) indicating the failure return code. In some embodiments, if an error has not occurred, at 810, the cognitive analytics pipeline worker or error handler component 202 can set a success return code and at 812, the cognitive analytics pipeline worker or error handler component 202 can reply to task allocation service (e.g., task manager component 108) indicating the success return code.

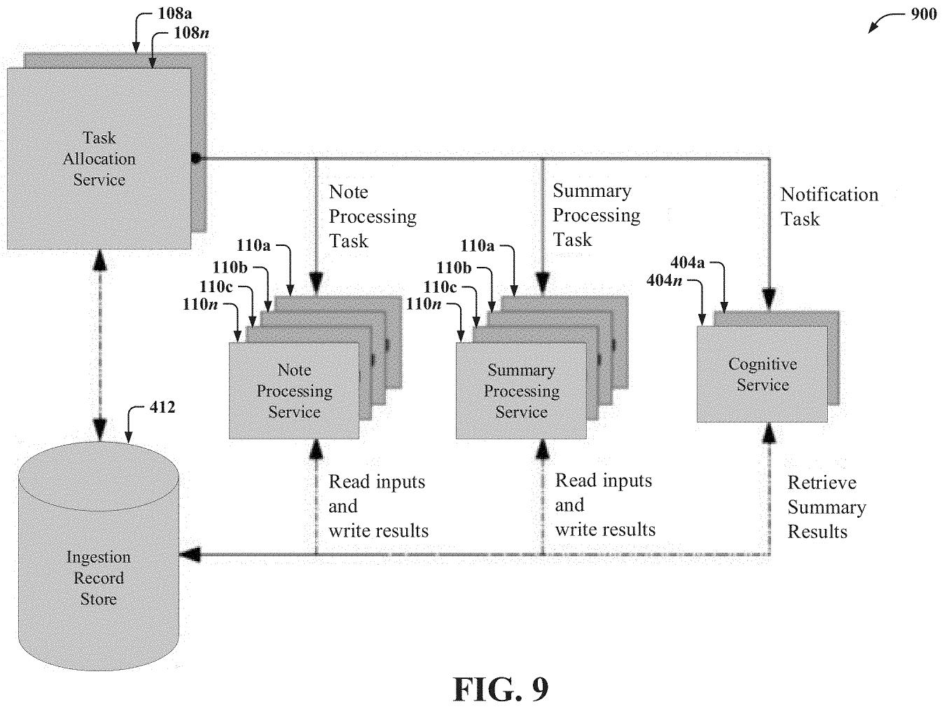

FIG. 9 illustrates a block diagram of an example, non-limiting system 900 that can facilitate scheduling processing and summarization of an electronic medical record based on a summarization deadline in accordance with one or more embodiments described herein. In some embodiments, system 900 can comprise an example, non-limiting alternative embodiment of system 100, system 200, system 300, system 400, or EMR processing and summarization system 102. In some embodiments, FIG. 9 can illustrate the distributed task management components of the subject disclosure in accordance with one or more embodiments described herein. Repetitive description of like elements or processes employed in respective embodiments is omitted for sake of brevity.

In some embodiments, system 900 can comprise one or more task manager components 108a, 108n (where n represents a total quantity of task manager components 108), which are denoted as task allocation service in FIG. 9. In some embodiments, system 900 can comprise one or more cognitive analysis components 110a, 110b, 110c, 110n (where n represents a total quantity of cognitive analysis components 110), which are denoted as note processing service and summary processing service in FIG. 9. In some embodiments, system 900 can comprise one or more cognitive service 404a, 404n (where n represents a total quantity of cognitive service 404).

In some embodiments, requests (e.g., EMR processing request, summary request, etc.) can be received by one or more task manager components 108a, 108n (e.g., via interface component 204, REST 302, cognitive service 404, etc.). In some embodiments, such one or more task manager components 108a, 108n can record the requests in incremental ingestion record store 412 (e.g., denoted ingestion record store in FIG. 9). In some embodiments, such one or more task manager components 108a, 108n can deconstruct the requests into discrete processing units (e.g., work items) and schedule such discrete processing units for processing (e.g., via a heuristics algorithm that can segment an EMR into data bundles as described above with reference to FIG. 1). In some embodiments, one or more cognitive analysis components 110a, 110b, 110c, 110n (e.g., distributed processors) can contact one or more task manager components 108a, 108n when they have capacity to process work items. In some embodiments, successfully processed work items (e.g., successfully annotated work items) can be recorded (e.g., by task manager components 108a, 108n or cognitive services 404a, 404n) in incremental ingestion record store 412 or cognitive record store 416.

In some embodiments, as described above, system 900 or EMR processing and summarization system 102 can facilitate parallel processing of one or more EMRs or one or more data bundles (e.g., data bundles 306a, 306b, 306c, 306d, 306n) of such EMRs. For example, as described above, one or more task manager components 108a, 108n of system 900 can distribute one or more tasks (e.g., note processing task, summary processing task, etc.) to a plurality of cognitive analysis components 110a, 110b, 110c, 110n (e.g., distributed processors), which can each process such one or more tasks independently of other tasks processed by other cognitive analysis components 110a, 110b, 110c, 110n. In this example, by facilitating such distributed processing, system 900 or EMR processing and summarization system 102 can thereby improve processing time to process such one or more EMRs or one or more data bundles (e.g., data bundles 306a, 306b, 306c, 306d, 306n). In this example, by facilitating such distributed processing, system 900 or EMR processing and summarization system 102 can thereby also improve processing performance of a processing unit associated with EMR processing and summarization system 102 (e.g., processor 106).

In some embodiments, EMR processing and summarization system 102 can be associated with various technologies. For example, EMR processing and summarization system 102 can be associated with EMR processing technologies, EMR summarization technologies, EMR cognitive analysis technologies, machine learning technologies, artificial intelligence technologies, task management technologies, data analytics technologies, cloud computing technologies, computer technologies, server technologies, information technology (IT) technologies, internet-of-things (IoT) technologies, automation technologies, data exchange technologies, or other technologies.

In some embodiments, EMR processing and summarization system 102 can provide technical improvements to systems, devices, components, operational steps, or processing steps associated with the various technologies identified above. For example, EMR processing and summarization system 102 can segment an EMR into one or more discrete data units (e.g., data bundles) that can be processed independently by one or more cognitive analytics pipelines (e.g., distributed processors), thereby improving processing time to independently process or summarize multiple EMRs corresponding to different patients and reducing latency associated with EMR processing and summarization technologies.

In some embodiments, EMR processing and summarization system 102 can provide technical improvements to a processing unit (e.g., processor 106, a CPU, etc.) associated with one or more of the various technologies identified above. For example, as described above, by facilitating distributed processing (e.g., via cognitive analysis component 110, distributed processors, etc.), EMR processing and summarization system 102 can thereby improve processing performance of a processing unit associated with EMR processing and summarization system 102 (e.g., processor 106) by reducing the processing workload of such a processing unit.

In some embodiments, EMR processing and summarization system 102 can employ hardware or software to solve problems that are highly technical in nature, that are not abstract and that cannot be performed as a set of mental acts by a human. In some embodiments, some of the processes described herein may be performed by one or more specialized computers (e.g., one or more specialized processing units, a specialized computer with automated diagnostics or optimization component(s), etc.) for carrying out defined tasks related to the various technologies identified above. In some embodiments, EMR processing and summarization system 102 or components thereof, can be employed to solve new problems that arise through advancements in technologies mentioned above, employment of cloud-computing systems, computer architecture, or another technology.

It is to be appreciated that EMR processing and summarization system 102 can utilize various combinations of electrical components, mechanical components, and circuitry that cannot be replicated in the mind of a human or performed by a human, as the various operations that can be executed by EMR processing and summarization system 102 or components thereof as described herein are operations that are greater than the capability of a human mind. For instance, the amount of data processed, the speed of processing such data, or the types of data processed by EMR processing and summarization system 102 over a certain period of time can be greater, faster, or different than the amount, speed, or data type that can be processed by a human mind over the same period of time.

According to several embodiments, EMR processing and summarization system 102 can also be fully operational towards performing one or more other functions (e.g., fully powered on, fully executed, etc.) while also performing the various operations described herein. It should be appreciated that such simultaneous multi-operational execution is beyond the capability of a human mind. It should also be appreciated that EMR processing and summarization system 102 can include information that is impossible to obtain manually by an entity, such as a human user. For example, the type, amount, or variety of information included in task manager component 108, cognitive analysis component 110, error handler component 202, or interface component 204 can be more complex than information obtained manually by a human user.

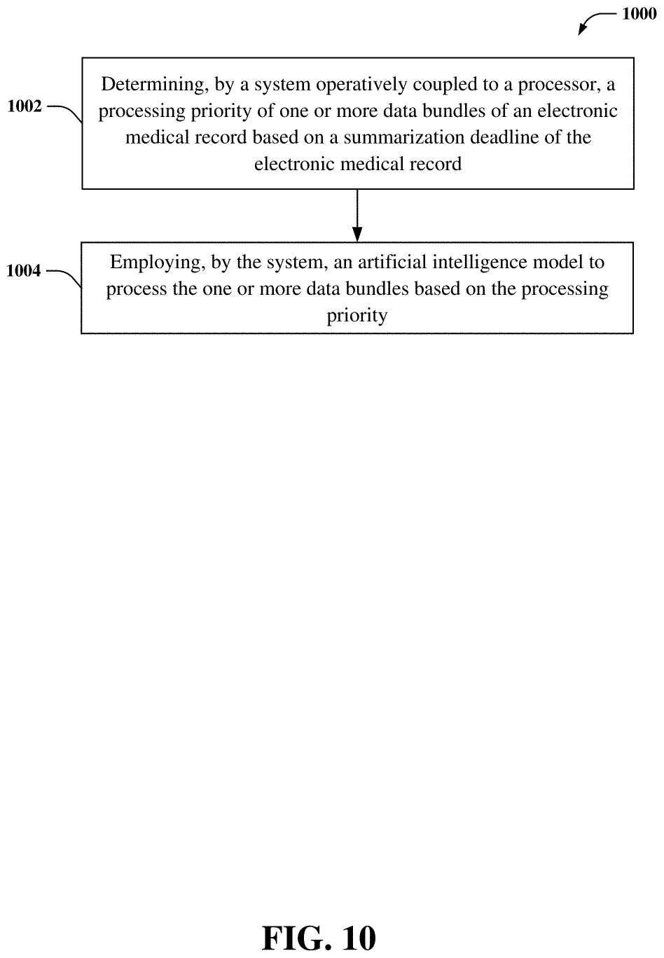

FIG. 10 illustrates a flow diagram of an example, non-limiting computer-implemented method 1000 that can facilitate scheduling processing and summarization of an electronic medical record based on a summarization deadline in accordance with one or more embodiments described herein. Repetitive description of like elements or processes employed in respective embodiments is omitted for sake of brevity.

At 1002, determining, by a system (e.g., via EMR processing and summarization system 102 or task manager component 108) operatively coupled to a processor (e.g., processor 106), a processing priority of one or more data bundles (e.g., data bundles 306a, 306b, 306c, 306d, 306n) of an electronic medical record based on a summarization deadline of the electronic medical record. At 1004, employing, by the system (e.g., via EMR processing and summarization system 102 or cognitive analysis component 110), an artificial intelligence model to process the one or more data bundles based on the processing priority.

For simplicity of explanation, the computer-implemented methodologies are depicted and described as a series of acts. It is to be understood and appreciated that the subject innovation is not limited by the acts illustrated or by the order of acts, for example acts can occur in various orders or concurrently, and with other acts not presented and described herein. Furthermore, not all illustrated acts can be required to implement the computer-implemented methodologies in accordance with the disclosed subject matter. In addition, those skilled in the art will understand and appreciate that the computer-implemented methodologies could alternatively be represented as a series of interrelated states via a state diagram or events. Additionally, it should be further appreciated that the computer-implemented methodologies disclosed hereinafter and throughout this specification are capable of being stored on an article of manufacture to facilitate transporting and transferring such computer-implemented methodologies to computers. The term article of manufacture, as used herein, is intended to encompass a computer program accessible from any computer-readable device or storage media.

In order to provide a context for the various aspects of the disclosed subject matter, FIG. 11 as well as the following discussion are intended to provide a general description of a suitable environment in which the various aspects of the disclosed subject matter can be implemented. FIG. 11 illustrates a block diagram of an example, non-limiting operating environment in which one or more embodiments described herein can be facilitated. Repetitive description of like elements or processes employed in other embodiments described herein is omitted for sake of brevity.

With reference to FIG. 11, a suitable operating environment 1100 for implementing various aspects of this disclosure can also include a computer 1112. The computer 1112 can also include a processing unit 1114, a system memory 1116, and a system bus 1118. The system bus 1118 couples system components including, but not limited to, the system memory 1116 to the processing unit 1114. The processing unit 1114 can be any of various available processors. Dual microprocessors and other multiprocessor architectures also can be employed as the processing unit 1114. The system bus 1118 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1394), and Small Computer Systems Interface (SCSI).

The system memory 1116 can also include volatile memory 1120 and nonvolatile memory 1122. The basic input/output system (BIOS), containing the basic routines to transfer information between elements within the computer 1112, such as during start-up, is stored in nonvolatile memory 1122. Computer 1112 can also include removable/non-removable, volatile/non-volatile computer storage media. FIG. 11 illustrates, for example, a disk storage 1124. Disk storage 1124 can also include, but is not limited to, devices like a magnetic disk drive, floppy disk drive, tape drive, Jaz drive, Zip drive, LS-100 drive, flash memory card, or memory stick. The disk storage 1124 also can include storage media separately or in combination with other storage media. To facilitate connection of the disk storage 1124 to the system bus 1118, a removable or non-removable interface is typically used, such as interface 1126. FIG. 11 also depicts software that acts as an intermediary between users and the basic computer resources described in the suitable operating environment 1100. Such software can also include, for example, an operating system 1128. Operating system 1128, which can be stored on disk storage 1124, acts to control and allocate resources of the computer 1112.

System applications 1130 take advantage of the management of resources by operating system 1128 through program modules 1132 and program data 1134, e.g., stored either in system memory 1116 or on disk storage 1124. It is to be appreciated that this disclosure can be implemented with various operating systems or combinations of operating systems. A user enters commands or information into the computer 1112 through input device(s) 1136. Input devices 1136 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1114 through the system bus 1118 via interface port(s) 1138. Interface port(s) 1138 include, for example, a serial port, a parallel port, a game port, and a universal serial bus (USB). Output device(s) 1140 use some of the same type of ports as input device(s) 1136. Thus, for example, a USB port can be used to provide input to computer 1112, and to output information from computer 1112 to an output device 1140. Output adapter 1142 is provided to illustrate that there are some output devices 1140 like monitors, speakers, and printers, among other output devices 1140, which require special adapters. The output adapters 1142 include, by way of illustration and not limitation, video and sound cards that provide a means of connection between the output device 1140 and the system bus 1118. It should be noted that other devices or systems of devices provide both input and output capabilities such as remote computer(s) 1144.

Computer 1112 can operate in a networked environment using logical connections to one or more remote computers, such as remote computer(s) 1144. The remote computer(s) 1144 can be a computer, a server, a router, a network PC, a workstation, a microprocessor based appliance, a peer device or other common network node and the like, and typically can also include many or all of the elements described relative to computer 1112. For purposes of brevity, only a memory storage device 1146 is illustrated with remote computer(s) 1144. Remote computer(s) 1144 is logically connected to computer 1112 through a network interface 1148 and then physically connected via communication connection 1150. Network interface 1148 encompasses wire or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 1150 refers to the hardware/software employed to connect the network interface 1148 to the system bus 1118. While communication connection 1150 is shown for illustrative clarity inside computer 1112, it can also be external to computer 1112. The hardware/software for connection to the network interface 1148 can also include, for exemplary purposes only, internal and external technologies such as, modems including regular telephone grade modems, cable modems and DSL modems, ISDN adapters, and Ethernet cards.

Referring now to FIG. 12, an illustrative cloud computing environment 1250 is depicted. As shown, cloud computing environment 1250 includes one or more cloud computing nodes 1210 with which local computing devices used by cloud consumers, such as, for example, personal digital assistant (PDA) or cellular telephone 1254A, desktop computer 1254B, laptop computer 1254C, or automobile computer system 1254N may communicate. Nodes 1210 may communicate with one another. They may be grouped (not shown) physically or virtually, in one or more networks, such as Private, Community, Public, or Hybrid clouds as described hereinabove, or a combination thereof. This allows cloud computing environment 1250 to offer infrastructure, platforms or software as services for which a cloud consumer does not need to maintain resources on a local computing device. It is understood that the types of computing devices 1254A-N shown in FIG. 12 are intended to be illustrative only and that computing nodes 1210 and cloud computing environment 1250 can communicate with any type of computerized device over any type of network or network addressable connection (e.g., using a web browser).

Referring now to FIG. 13, a set of functional abstraction layers provided by cloud computing environment 1250 (FIG. 12) is shown. It should be understood in advance that the components, layers, and functions shown in FIG. 13 are intended to be illustrative only and embodiments of the invention are not limited thereto. As depicted, the following layers and corresponding functions are provided:

Hardware and software layer 1360 includes hardware and software components. Examples of hardware components include: mainframes 1361; RISC (Reduced Instruction Set Computer) architecture based servers 1362; servers 1363; blade servers 1364; storage devices 1365; and networks and networking components 1366. In some embodiments, software components include network application server software 1367 and database software 1368.

Virtualization layer 1370 provides an abstraction layer from which the following examples of virtual entities may be provided: virtual servers 1371; virtual storage 1372; virtual networks 1373, including virtual private networks; virtual applications and operating systems 1374; and virtual clients 1375.

In one example, management layer 1380 may provide the functions described below. Resource provisioning 1381 provides dynamic procurement of computing resources and other resources that are utilized to perform tasks within the cloud computing environment. Metering and Pricing 1382 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 1383 provides access to the cloud computing environment for consumers and system administrators. Service level management 1384 provides cloud computing resource allocation and management such that required service levels are met. Service Level Agreement (SLA) planning and fulfillment 1385 provide pre-arrangement for, and procurement of, cloud computing resources for which a future requirement is anticipated in accordance with an SLA.

Workloads layer 1390 provides examples of functionality for which the cloud computing environment may be utilized. Non-limiting examples of workloads and functions which may be provided from this layer include: mapping and navigation 1391; software development and lifecycle management 1392; virtual classroom education delivery 1393; data analytics processing 1394; transaction processing 1395; and electronic medical record (EMR) processing and summarization software 1396.

The present invention may be a system, a method, an apparatus or a computer program product at any possible technical detail level of integration. The computer program product can include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, Java, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations or block diagrams of methods, apparatus (systems), and computer program products according to embodiments of the invention. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer executable components; and
a processor that executes the computer executable components stored in the memory, wherein the computer executable components comprise:
a task manager component that determines a processing priority of one or more data bundles of an electronic medical record based on a summarization deadline of the electronic medical record; and
a cognitive analysis component that employs an artificial intelligence model to process the one or more data bundles based on the processing priority.

2. The system of claim 1, wherein the task manager component further employs a heuristic algorithm to segment the electronic medical record into the one or more data bundles.

3. The system of claim 1, wherein the task manager component further employs a scheduling algorithm to schedule processing of the one or more data bundles based on the processing priority.

4. The system of claim 1, wherein the task manager component further employs a scheduling algorithm to schedule summarization of the electronic medical record based on at least one of: the summarization deadline; one or more processed data bundles; a processed partial electronic medical record; or a processed full electronic medical record.

5. The system of claim 1, wherein the task manager component further distributes at least one of the one or more data bundles or a summarization request to summarize the electronic medical record to one or more distributed processors, thereby facilitating improved processing performance of the processor and scale out ability of the system to process data bundles or summarization requests in parallel.

6. The system of claim 1, wherein the task manager component further tracks at least one of processing status of the one or more data bundles or summarization status of the electronic medical record.

7. The system of claim 1, wherein the computer executable components further comprise an error handler component that:
detects at least one of a data error or a processing error;
corrects at least one of the data error or the processing error; and
employs a scheduling algorithm to schedule processing of at least one of a corrected data bundle, the one or more data bundles, or a summarization of the electronic medical record based on at least one of a corrected data error, a corrected processing error, the processing priority, or the summarization deadline.

8. The system of claim 1, wherein the cognitive analysis component employs at least one of a reasoning algorithm, natural language annotation, or natural language processing to perform at least one of data extraction or data annotation of data in the one or more data bundles.

9. The system of claim 1, wherein the computer executable components further comprise an interface component that:
receives at least one of the electronic medical record or a summarization request to summarize the electronic medical record; and
provides processing status of the summarization request, and wherein the processing status comprises a completed status, a partially completed status, or a failure status.

10. The system of claim 1, wherein the memory can further store at least one of intermediate processing results or final processing results of at least one of the one or more data bundles or a summarization request to summarize the electronic medical record, and wherein the system utilizes at least one of the intermediate processing results or the final processing results to process at least one of a subsequent electronic medical record or a subsequent summarization request.

11. The system of claim 1, wherein the task manager component further revises the processing priority of at least one data bundle in processing based on a revised summarization deadline.

12. A computer-implemented method, comprising:
determining, by a system operatively coupled to a processor, a processing priority of one or more data bundles of an electronic medical record based on a summarization deadline of the electronic medical record; and
employing, by the system, an artificial intelligence model to process the one or more data bundles based on the processing priority.

13. The computer-implemented method of claim 12, further comprising:
employing, by the system, a heuristic algorithm to segment the electronic medical record into the one or more data bundles.

14. The computer-implemented method of claim 12, further comprising:
employing, by the system, a scheduling algorithm to schedule at least one of:
processing of the one or more data bundles based on the processing priority; or
summarization of the electronic medical record based on at least one of the summarization deadline, one or more processed data bundles, a processed partial electronic medical record, or a processed full electronic medical record.

15. The computer-implemented method of claim 12, further comprising:
distributing, by the system, at least one of the one or more data bundles or a summarization request to summarize the electronic medical record to one or more distributed processors, thereby facilitating improved processing performance of the processor and scale out ability of the system to process data bundles or summarization requests in parallel.

16. The computer-implemented method of claim 12, further comprising:
detecting, by the system, at least one of a data error or a processing error;
correcting, by the system, at least one of the data error or the processing error; and
employing, by the system, a scheduling algorithm to schedule processing of at least one of a corrected data bundle, the one or more data bundles, or a summarization of the electronic medical record based on at least one of a corrected data error, a corrected processing error, the processing priority, or the summarization deadline.

17. A computer program product facilitating scheduling processing and summarization of an electronic medical record based on a summarization deadline, the computer program product comprising a computer readable storage medium having program instructions embodied therewith, the program instructions executable by a processor to cause the processor to:
determine, by the processor, a processing priority of one or more data bundles of an electronic medical record based on a summarization deadline of the electronic medical record; and
employ, by the processor, an artificial intelligence model to process the one or more data bundles based on the processing priority.

18. The computer program product of claim 17, wherein the program instructions are further executable by the processor to cause the processor to:
employ, by the processor, a heuristic algorithm to segment the electronic medical record into the one or more data bundles.

19. The computer program product of claim 17, wherein the program instructions are further executable by the processor to cause the processor to:
employ, by the processor, a scheduling algorithm to schedule at least one of:
processing of the one or more data bundles based on the processing priority; or
summarization of the electronic medical record based on at least one of the summarization deadline, one or more processed data bundles, a processed partial electronic medical record, or a processed full electronic medical record.

20. The computer program product of claim 17, wherein the program instructions are further executable by the processor to cause the processor to:
distribute, by the processor, at least one of the one or more data bundles or a summarization request to summarize the electronic medical record to one or more distributed processors.
